(19) United States
(12) Patent Application Publication
(10) Pub. No.: US 2010/0292449 A1
(43) Pub. Date: Nov. 18, 2010

(54) WATER-STABLE COMPOUNDS, CATALYSTS AND CATALYSED REACTIONS

(75) Inventors: Arran Alexander Dickon Tulloch, Northallerton (GB); Alan Cooper, Wingate (GB); Robert Hume Duncan, Stockton on Tees (GB)

Correspondence Address: RATNERPRESTIA, P.O. BOX 980, VALLEY FORGE, PA, 19482, US

(73) Assignee: Johnson Matthey PLC, London (GB)

(21) Appl. No.: 12/665,387

(22) PCT Filed: Jun. 16, 2008
(86) PCT No.: PCT/GB08/50450

§ 371 Date: Jun. 23, 2010

(30) Foreign Application Priority Data
Jun. 18, 2007 (GB) 0711733.6
Nov. 27, 2007 (GB) 0723162.4
Jan. 8, 2008 (GB) 0800257.8

(51) Int. Cl. C07C 291/08; B01J 31/12; C07C 69/80

(52) U.S. Cl. 534/572; 502/155; 560/76

(57) ABSTRACT

The invention provides a method of carrying out a Lewis-acid catalysed organic reaction in the presence of a Lewis acid catalyst comprising a metal-organic compound having the following formula: M(HO(CR¹R²)z)a(O(CR¹R²)z)bY—(CR³R⁴)χ-Y((CR¹R²)zO)c((CR¹R²)zOH)d.nR⁵OH (Formula I) in which: M is a metal atom, preferably titanium, zirconium, hafnium or iron (III) Y is selected from P and N, but Ss very preferably N; each R¹, R², R³ and R⁴ is independently selected from H, alkyl, aryl, substituted alkyl or substituted aryl, R⁵ is hydrogen, an alkyl group, a hydroxy-functionalised alkyl group, a polyoxyalkylmoiety, R⁶O or R⁷COO where R⁶ and R⁷ may each represent H, alkyl, aryl or alkyl-aryl; d and a are each O or 1, b and c are each 1 or 2, b+c=the valency of M, a+b+c+d=4, each z is independently 1, 2, 3 or 4; x represents the least number of C atoms between the Y atoms and is 2 or 3 and n is a number in the range from 0 to 4. The metal-organic compound forms a stable hydrate in water which retains Lewis-acid catalytic properties.

The present invention concerns metal-organic compounds, in particular metal chelate compounds having a new ligand composition which are stable in contact with water and which have Lewis acidic properties. The compounds are useful in a range of Lewis acid-catalysed organic reactions, especially such reactions in which water may be present.

Metal-organic compounds formed by reacting metal compounds with organic compounds having a hydroxyl group are very well known. Metal alkoxides and beta-diketonates in particular, such as titanium tetraisopropoxide and titanium acetylacetonate for example, have been known and used in industrial applications for many years. The reaction of titanium compounds with alkanolamines has also been used to provide stable chelates. For example, GB-A-2207426 describes the use as a thixotropic agent in aqueous emulsion paints of a titanium chelate which is the reaction product of a titanium orthoester, a glycol or glycol ether, an alkanolamine and an alpha-hydroxy carboxylic acid which is a hydroxy mono-carboxylic acid or a hydroxy dicarboxylic acid. Verkade et al (Y. Kim and J. G. Verkade, Organometallics (2002), 21, 2395-2399) describe titanatranes formed by the reaction of tetra(isopropyl)titanate with 2,6-di-isopropylphenol and either tris(2-hydroxy-3,5-dimethylbenzyl)amine or triethanolamine or a tertiary amine having a combination of 2-hydroxy-3,5-dimethylbenzyl- and hydroxyethyl-substituents. Tshuva et al (Dalton Trans., (2006) 4169-4172) have studied hydroxylamine complexes of titanium, particularly to investigate their potential as hydrolytically stable forms of active titanium compounds. They found that the complexes formed were relatively stable in water at pH=5 but decomposed over a few hours at higher pH. EP-A-0368911 describes compounds of titanium formed by the reaction of a titanium tetraalkoxide with a dialkanolamine in a 1:1 mole ratio, followed by controlled hydrolysis of the resulting product. The compounds are described as stable in water and active as catalysts for esterification reactions.

Lewis acids are important catalysts used in many organic reactions but have the major disadvantage that they are usually highly reactive to water and therefore may be difficult to use in reactions where water is present. Kobayashi et al (J. Am. Chem. Soc. (1998) 120, 8287-8288) describe new water stable Lewis acids which are rare earth metal triflates and Kobayashi and Manabe (Pure Appl. Chem., vol 72, No 7, 1373-1380 (2000)) and U.S. Pat. No. 6,525,227 discuss their use as “green” Lewis acid catalysts for organic synthesis reactions. There is, however, a need for alternative compounds which are stable in water and which are useful as economical and fluorine-free Lewis acid catalysts.

EP-A-0278684 describes water-soluble zirconium chelates formed by the reaction of zirconium tetraalkoxide with N-(2-hydroxyethyl)-N-(2-hydroxypropyl)-N′,N′-bis-(2-hydroxypropyl)ethylenediamine as cross-linkers in hydraulic fracturing fluids. U.S. Pat. No. 2,824,115 describes organometallic compounds which are esters of titanium or zirconium and aminoalcohols, including “Quadrol” (N,N,N′,N′-tetrakis(2-hydroxypropyl)ethylenediamine), and their use as dispersing agents, paint additives, treating agents for wool and other fibres and in cosmetic applications. U.S. Pat. No. 3,294,689 describes the use of N,N,N′,N′-tetrakis(2-hydroxyethyl)ethylenediamine and similar polyhydroxyamines as a component of a sequestering agent for Fe, Mn, Cu, Zn and Ni ions. None of the prior art references describe the use of the metal-organic compounds described herein as catalysts.

It is an object of the invention to provide new metal-organic compounds. It is a further object of the invention to provide a hydrolytically stable metal complex which is useful in catalysis and to provide a method of carrying out a Lewis acid-catalysed reaction.

According to the invention we provide a catalyst comprising a metal-organic compound having the formula:

M(HO(CR¹R²)_{z})ₐ(O(CR¹R²)_{z})_{b}Y—(CR³R⁴)ₓ—Y((CR¹R²)_{z}O)_{c}((CR¹R²)_{z}OH)_{d}.nR⁵OH (Formula I)

in which:
M is a metal atom
Y is selected from P and N, but is very preferably N;
each R¹, R², R³ and R⁴ is independently selected from H, alkyl, aryl, substituted alkyl or substituted aryl,
R⁵ is hydrogen, an alkyl group, a hydroxy-functionalised alkyl group, a polyoxyalkylmoiety, R⁶O or R⁷COO where R⁶ and R⁷ may each represent H, alkyl, aryl or alkyl-aryl;
d and a are each 0 or 1,
b and c are each 1 or 2,
b+c=the valency of M,
a+b+c+d=4,
each z is independently 1, 2, 3 or 4;
x represents the least number of C atoms between the Y atoms and is 2 or 3 and
n is a number in the range from 0 to 4.

The metal-organic compound of Formula I has Lewis-acidic properties and is useful as a Lewis-acid catalyst because of its stability in water and polar alcohols. An important aspect of the invention is therefore found in the use of a metal-organic compound having the general formula shown in Formula I as a catalyst for a chemical reaction, including but not limited to a reaction to form one or more single or multiple bonds between carbon and carbon, carbon and oxygen, carbon and nitrogen, oxygen and nitrogen, oxygen and sulphur and/or nitrogen and nitrogen atoms, useful in organic synthesis. Such reactions include aldol reactions, Michael addition, Mannich reaction, esterification, ether formation, oxidation, oxidative coupling, peptide synthesis, amide synthesis, Claisen reactions and condensation reactions such as polymerisation.

According to a further aspect of the invention, we provide a composition comprising:

from 0.01-70% by weight of a Lewis acid catalyst of Formula I and from 0.1-99.99% by weight of water, or an alcohol or a mixture thereof, the balance comprising one or more organic compounds.

The composition may take the form of a feedstock, catalyst, reaction mixture or product of a Lewis-acid catalysed reaction. The metal-organic compound may be dissolved in the water or alcohol or mixture thereof. Other solvents may also be present. The Lewis acid catalyst may be dissolved in any suitable solvent.

In a still further aspect of the invention we provide a method of carrying out a catalysed reaction comprising carrying out the reaction in the presence of a catalyst comprising a metal-organic compound having the general formula of Formula I.

In a still further aspect of the invention we provide a method of carrying out a Lewis-acid catalysed organic reaction wherein the composition of the invention is present as a feedstock, catalyst, reaction mixture or product.

The metal M is selected from any metal capable of forming a covalent metal-oxygen bond. Preferred metals include titanium, zirconium, hafnium, iron (III) aluminium and tin, especially titanium, zirconium, hafnium and iron (III). Particularly preferred metals include titanium and zirconium, especially titanium.

Y represents nitrogen or phosphorus but is most preferably a nitrogen atom. The Y atom is capable of forming a co-ordinate bond with the metal to stabilise the complex. Without wishing to be bound by theory, it is believed that the electronic structure of N is particularly susceptible to the formation of such bonds in the complex.

Each R¹ and R², may be the same as or different from each other R¹ and/or R². This means also that in the (HO(CR¹R²)_{z})₂— part of Formula I, each of the two (CR¹R²)_{z} moieties may be the same or different. R¹ and R² may be selected from H, alkyl, aryl, substituted alkyl or substituted aryl. When R¹ and/or R² is an alkyl or substituted alkyl, the alkyl group preferably contains from 1 to 12, more preferably from 1 to 8 carbon atoms and may be linear or branched. When R¹ and/or R² is an aryl or substituted aryl group then it is preferably a phenyl group, or a substituted phenyl. The group —(CR¹R²)_{z}— may form a part of a larger structure, such as an aryl or cyclo-alkyl ring for example, and in such cases R¹ and R² may be linked to each other or to another CR¹R² moiety when z>1. Any of the CR¹R² moieties may form part of a polymeric structure, such as a vinyl polymer for example, or form a part of a pendant group attached to a polymeric molecule. In preferred embodiments, each one of R¹ and R² is either a hydrogen atom, a methyl or an ethyl group.

R³ and R⁴ may be the same as or different from each other. They may be selected from H, alkyl, aryl, substituted alkyl or substituted aryl and may be selected from the same groups described in relation to R¹ and R². R³ and R⁴ may be the same as or different from R¹ and/or R². —(CR³R⁴)ₓ— is a bridging group between the two Y atoms. X represents the number of C atoms between the two Y atoms and is preferably 2 or 3 so that when the Y atoms each form a co-ordinate bond the metal, Y atoms and bridging group —(CR³R⁴)ₓ— together form a 5- or 6-membered ring. The bridging group —(CR³R⁴)_{X}— may form a part of a larger structure, such as an aryl or cycloalkyl ring for example and in such cases R³ and R⁴ may be linked to each other or to another CR³R⁴ moiety when x>1. Any of the CR³R⁴ moieties may form part of a polymeric structure, such as a vinyl polymer for example, or form a part of a pendant group attached to a polymeric molecule. In one preferred embodiment each one of R³ and R⁴ is a hydrogen atom or a methyl group, and is more preferably a hydrogen atom.

By appropriate selection of R¹, R², R³ and R⁴, the compound may be chiral at one or more of the CR¹R² or CR³R⁴ carbon atoms.

Each z is 1, 2, 3 or 4 and may be the same as or different from each other z. Preferably z is at least 2 and more preferably z is 2 or 3. When z is 2 or 3 the metal, each —O(CR¹R²)_{z} moiety and a Y atom may together form a 5- or 6-membered ring in the metal-organic compound.

The metal organic compound of the invention is a chelate formed by the reaction of a chelating compound of Formula II with a compound of the metal M:

(HO(CR¹R²)_{z})₂Y—(CR³R⁴)ₓ—Y((CR¹R²)_{z}OH)₂ (Formula II)

When metal M has a valency of 4, any or all of the four hydroxyl groups may react with the metal to form a metal oxygen covalent bond. In this case, in Formula I, b and c are each 2 and d and a are both 0. When the valency of M is less than 4, not all of the hydroxyl groups can react at any one time and therefore there may be unreacted hydroxyl groups present in the chelate. These hydroxyl groups may, however, form co-ordinate bonds with metal M and therefore participate in stabilising the chelate. When M is a trivalent metal, in Formula I, a=1, b=1, c=2 and d=0.

A preferred chelating compound comprises (HO(CH₂)₂)₂N—(CH₂)₂—N((CH₂)₂OH)₂ i.e. N,N,N′,N′-tetrakis(2-hydroxyethyl)ethylenediamine, which may be known as and designated herein as THEED. In one preferred embodiment, the metal organic compound comprises N,N,N′,N′-tetrakis(2-ethoxy)ethylenediamine titanium Ti(TOEED). This is believed to be a new compound. This compound is very stable to hydrolysis and so may be used as a catalyst for reactions in which water is present. A second preferred chelating compound comprises (HOCH(CH₃)CH₂)₂N—(CH₂)₂—N(CH₂CH(CH₃)OH)₂ i.e. N,N,N′,N′-tetrakis(2-hydroxypropyl)ethylenediamine, which may be known as and designated herein as THPED. A preferred catalyst formed from THPED is N,N,N′N′-tetrakis-(2-hydroxypropyl)ethylenediamine titanium (which may be known and designated herein as Ti(TOPED)).

The value of n depends on the oxidation state of the metal and its coordination number. n=1 when M is a metal such as titanium, which has an oxidation state of 4 and is believed to be 7-coordinate in the compounds described. When M is a metal such as zirconium or hafnium, having an oxidation state of 4 and a coordination number of 8, then n=from 1-3, normally 1 or 2.

When n>0, R⁵OH is coordinated to the metal chelate and is derived from a solvent, reactant or other molecule present in a mixture with the metal chelate. Such a mixture in equilibrium may include one or more molecules of the metal chelate in which R⁵OH is present as R⁵⁰— and covalently bonded to the metal, displacing one or more of the hydroxyl groups of the chelating compound. For example a solution of N,N,N′,N′-tetrakis(2-ethoxy)ethylenediamine titanium in methanol may include various species of the type: (OCH₂CH₂)₂N—(CH₂)₂—N—CH₂CH₂OH(CH₂CH₂O)—Ti—OCH₃. For simplicity, however, we will denote solvating or coordinating molecules as R⁵OH herein.

R⁵ is hydrogen, an alkyl group or a hydroxy-functionalised alkyl group or a polyoxyalkylmoiety when R⁵OH represents water, an alkyl alcohol or a diol or polyol. Preferred hydrated compounds, i.e. where R⁵OH is water, include N,N,N′,N′-tetrakis(2-ethoxy)ethylenediamine metal hydrate, and N,N,N′,N′-tetrakis(2-propoxy)ethylenediamine metal hydrate where the metal is selected from titanium, zirconium, hafnium and iron (III). The hydrated forms of the compound are particularly stable to hydrolysis and may be stored in contact with water for extended periods of time without significant loss of catalytic activity. The hydrated compound is formed when the non-hydrated compound is mixed with water. It is therefore also likely to be formed in situ when the compound is present in a reaction mixture with water. When R⁵OH is an alcohol (or a polyol, including a diol) then the alcohol coordinates to the metal, stabilising the complex. When water is present, the water-stabilised complex and the alcohol-stabilised complex exist in equilibrium. When a composition comprising a compound having the formula of Formula I is used as a catalyst for the activation of hydrogen peroxide, an organic hydroperoxide or a peroxyacid for the oxidation of a chemical substrate, it is likely that the metal-organic compound coordinates to a molecule of water or a solvent or to the peroxide or peroxyacid. When R⁵ is derived from a peroxide or hydroperoxide then R⁵ is R⁶O. When R⁵ is derived from a peroxyacid then R⁵ is R⁷COO where R⁶ and R⁷ may each represent H, alkyl, aryl or alkyl-aryl. It is likely that the hydrated (or otherwise solvated) forms of the complex and the peroxo-coordinated forms of the complex are both present when the complex is in a solution of a peroxide or peroxyacid.

The compounds may form stable solutions in water or alcohols up to relatively high concentrations, e.g. up to about 70% by weight of Ti(TOEED) in water at about 20° C. The aqueous solutions appear to be more stable at lower pH. For example, a 10% by weight aqueous solution of Ti(TOEED) is stable at pH 10 but starts to form a precipitate if the pH is raised to 11 or more. When the non-hydrated form is dimeric, as is believed to be the case for N,N,N′,N′-tetrakis(2-ethoxy)ethylenediamine titanium for example, then the dimer and the hydrate are in equilibrium when water is present.

The metal-organic compound may be prepared by mixing together a metal compound with the chelating compound with stirring. The reactants may be added in any order. Heating or cooling may be provided if required. For example, when the metal organic compound comprises N,N,N′,N′-tetrakis(2-ethoxy)ethylenediamine titanium Ti(TOEED) formed by the addition of the ligand compound to a titanium alkoxide, the reaction becomes quite hot. The heating may be controlled by mixing the components very slowly or by cooling the mixture. The co-product(s) from the reaction of the ligand-forming compound with the metal compound may be removed from the reaction mixture by suitable means such as distillation, derivitisation, or other separation means depending on the nature of the product. The co-product is e.g. a hydrogen halide or an alcohol when a metal halide or alkoxide is used as the starting metal compound. The co-product may alternatively be retained in the final product if desired. The reaction may take place in the presence of a suitable solvent if required.

The metal compound is capable of reacting with at least one of the hydroxyl groups present in the chelating compound to form a metal-oxygen bond. Suitable metal compounds include metal halides, metal alkoxides, metal halo-alkoxides, metal carboxylates and mixtures of these compounds. Typical alkoxides have the general formula M(OR)_{y} in which M is Ti, Zr, Hf, Al, Fe or Sn, y is the oxidation state of the metal, i.e. 3 or 4, and R is a substituted or unsubstituted, cyclic or linear, alkyl, alkenyl, aryl or alkyl-aryl group or mixtures thereof. Preferably, R contains up to 8 carbon atoms and, more preferably, up to 6 carbon atoms. Generally, all OR groups are identical but alkoxides derived from a mixture of alcohols can be used and mixtures of alkoxides can be employed when more than one metal is present in the complex. When the metal is titanium, preferred titanium compounds include titanium alkoxides having a general formula Ti(OR)₄ in which R is an alkyl group, preferably having from 1 to 8 carbon atoms and each R group may be the same as or different from the other R groups. Particularly suitable metal compounds include titanium tetrachloride, titanium tetra-isopropoxide, titanium tetra-n-propoxide, titanium tetra-n-butoxide, titanium tetraethoxide (tetraethyl titanate), zirconium n-propoxide, zirconium butoxide, hafnium butoxide, tin isopropoxide, tin butoxide, tin tetrachloride, tin tetrabromide, aluminium sec-butoxide, aluminium trichloride, iron(III)chloride, aluminium trimethoxide, iron trimethoxide, aluminium triethoxide, iron triethoxide, aluminium tri-isopropoxide, iron tri-isopropoxide, aluminium tri-n-propoxide, iron tri-n-propoxide, aluminium tritertiarybutoxide, iron tritertiarybutoxide, and iron tri-sec-butoxide.

The compounds of the invention may be used as catalysts in many Lewis acid catalysed organic reactions. The stability of the metal-organic compounds of the invention in water and alcohols allows their use in such reactions in which water is present, e.g. as a solvent or reactant. The availability of water as a solvent for a reaction when the Lewis acid catalysts of the invention are used clearly offers significant environmental advantages over the use of Lewis acid catalysts which are not stable to water. Furthermore, if water or an alcohol is produced during the reaction, or if traces of water may be present in the reaction mixture (e.g. by use of a “wet” solvent) or in the atmosphere under which such a reaction is carried out then the compounds of the invention may be used as water-tolerant Lewis-acid catalysts in such reactions without the risk of unwanted hydrolysis of the catalyst. Use of a compound of the invention as a catalyst also has the benefit that water may be used in the work-up of a reaction product mixture. For example, when the catalyst in a reaction is a water-stable compound of the invention, it may be separated from an organic reaction mixture by washing with water or an aqueous solution and optionally may then be reused. Typical alcohols which may be present in a composition comprising the Lewis acid catalyst are monohydric alcohols, especially C1-C8 alkyl alcohols such as methanol and ethanol; and polyhydric alcohols such as ethylene glycol, diethylene glycol and polyethylene glycols. The titanium catalyst, for example, is resistant to the formation of titanium methoxide in the presence of methanol and so offers a considerable benefit compared with the use of conventional titanium catalysts, such as titanium alkoxides. The high Lewis acid activity and high hydrolytic stability of the catalysts used in the methods of the invention combined with the non-flammable nature of the catalyst, make the catalyst highly desirable for many industrial reactions. Furthermore, the product of the reactions will avoid being contaminated by the labile alkoxy groups, released from standard metal alkoxide catalysts.

The catalysed reaction may comprise a reaction to form one or more single or multiple bonds between carbon and carbon, carbon and oxygen, carbon and nitrogen, oxygen and nitrogen, oxygen and sulphur and/or nitrogen and nitrogen atoms, useful in organic synthesis. Such reactions include aldol reactions, Michael addition, Mannich reaction, esterification, ether formation, oxidation, peptide synthesis, amide synthesis, Claisen reactions and condensation reactions such as polymerisation.

A process according to the invention for the oxidation of a chemical substrate comprises contacting the chemical substrate with hydrogen peroxide, an organic hydroperoxide or a peroxyacid and with a metal-organic compound of Formula I under conditions of temperature and pressure suitable to effect the desired oxidation reaction. Such a process is useful in various industrial processes such as chemical synthesis involving oxidations, such as N-oxidation, e.g. to form hydroxylamines, nitroso compounds, azoxy compounds and nitrones. Another important industrial process is the formation of peracids by the reaction of a peroxide, especially hydrogen peroxide with an acid, especially a carboxylic acid, e.g. acetic acid to form peracetic acid, which may then be used for the oxidation or peroxidation of oxidisable substrates such as unsaturated hydrocarbons, e.g. alkenes and alkynes to form epoxides. The epoxides thus formed may be hydrolysed or ring-opened with an alcohol to form diols. Bleaching is an important industrial process in which the use of hydrogen peroxide may provide significant environmental benefits. Such processes include the bleaching of wood and paper pulps, textile bleaching including the use in detergent formulations which have a bleaching action such as laundry detergents. The process may be used for the treatment of waste streams, e.g. municipal waste and the oxidation of sulphur-containing compounds such as H₂S, organic sulphides and cyclic sulphur compounds such as thiophenes. Industrial effluents may be treated using the process of the invention, for example to detoxify cyanide, nitrite and hypochlorite and for the removal of sulphite, thiosulphate and sulphide compounds.

A particularly important process of the invention is the oxidative coupling of aromatic amines to form azoxy compounds. Azoxy compounds are important for use as dyestuffs, in liquid crystal displays and other applications such as for therapeutic uses. The use of the process of the present invention, wherein a particular type of metal organic compound is used as a catalyst, enables the preparation of azoxy compounds from amines at selectivities >80% using water as a solvent. Surprisingly, the presence of water in the reaction mixture does not deactivate the catalyst, even when a titanium compound is used, and the catalyst remains active throughout several batches. The preparation of azoxy compounds may be carried out in-situ on a substrate which is to be dyed by the resulting coloured azoxy compound(s). Such applications include the dyeing of fibres and cloth and the colouration of human and animal hair and skin. In particular, the application of permanent hair colourants commonly involves the use of hydrogen peroxide and an activator. The peroxide has several functions in such a system, but an important function is the oxidative coupling of aromatic amines to form coloured species including azoxy compounds. The activation of hydrogen peroxide using the metal-organic composition of Formula I provides a water-stable oxidation system which avoids the use of ammonia. The activity and selectivity of the formation of azoxy compounds from aromatic amines using the process of the invention avoids the formation of by-products. WO-2006/106366 describes the use of titanium compounds in topical products for application to the skin and hair, including hair colourants, to improve the coupling between the body surface and the product. The use of the compound of Formula I in such products may further improve the performance of the product due to the inherent stability of the metal-organic compound in water.

The use of the catalyst of general Formula I in esterification reactions includes direct esterification, where an ester is formed by the reaction of an alcohol with a carboxylic acid or anhydride, such as, for example the reaction between phthalic acid and an alcohol such as 2-ethylhexanol to form dioctyl phthalate. Interesterification, in which two esters react with the exchange of alcohol residues and transesterification where an ester is reacted with an alcohol, such as the reaction of fats and oils, i.e. glycerides, with an alcohol such as methanol are also industrially important processes in which the catalyst of Formula I may be used.

The invention will be demonstrated in the following examples.

### EXAMPLE 1

### Preparation of Ti[TOEED]

236 g (1 mole) of N,N,N′,N′-tetrakis(2-hydroxyethyl)ethylenediamine (THEED) (from Sigma-Aldrich/Fluka) was added to 284 g (1 mole) of tetra(isopropoxy)titanium (VERTEC™ TIPT, from Johnson Matthey Catalysts) slowly and with stirring, to give a clear yellow solution. The isopropanol produced in the reaction was removed by rotary evaporation under reduced pressure to yield a pale yellow powder (280 g) of N,N,N′,N′-tetrakis(2-ethoxy)ethylenediamine titanium (Ti[TOEED]).

### EXAMPLE 2

The compound of Example 1 was dissolved in water to form a 10% w/w aqueous solution. The solution was boiled for one hour and then the water was removed by evaporation. The resulting pale yellow powder was found to be the same compound as the starting material, showing that the compound was stable to hydrolysis under the conditions used. The yellow powder was recrystallised from chloroform and analysed using ¹H-NMR, elemental analysis and a crystal structure determined by X-ray crystallography.

The NMR analysis yielded the following chemical shift data (relative to tetramethyl silane (TMS), where m indicates a multiplet, which is consistent with the presence of N,N,N′,N′-tetrakis(2-ethoxy)ethylenediamine titanium:

¹H NMR (400 MHz); 4.86-4.72 (2H, m), 4.72-4.60 (2H, m), 4.60-4.52 (1H, m), 4.52-4.43 (1H, m), 4.16-4.08 (1H, m), 4.08-4.01 (1H, m), 3.64-3.52 (2H, m), 3.43-3.31 (2H, m), 3.31-3.16 (2H, m), 3.12-3.01 (1H, m), 2.97-2.71 (5H, m).

The elemental analysis yielded the following data:

Found: C, 42.43; H, 7.19; N, 9.79%.

Theoretical for [Ti(TOEED)]₂: C, 42.87; H, 7.20; N, 10.00%.

Ti Content (wt %): Found: 16.98%, Theoretical for [Ti(TOEED)]₂: 17.08%

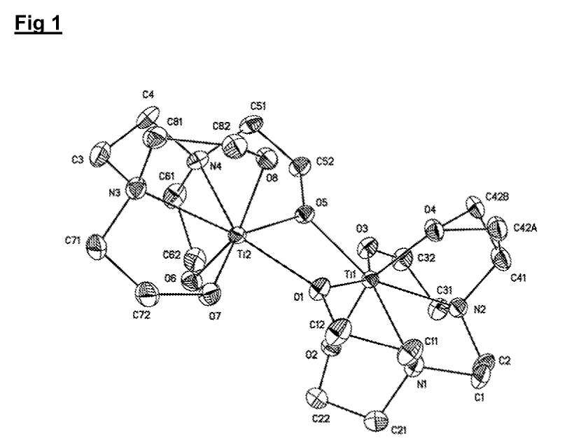

The crystal structure is presented in FIG. 1. The structure appears to be dimeric, having two Ti centres bridged by two oxygen atoms, designated O1 and O5 in the diagram.

### EXAMPLE 3

Example 1 was repeated except that the TIPT was added to the THEED. A similar pale yellow powder resulted.

### EXAMPLE 4

236 g (1 mole) of THEED was added to 284 g (1 mole) TIPT, slowly and with stirring, to give a clear yellow solution. 360 g of water was added to the solution and a mixture of water and isopropanol was removed by azeotropic distillation until all of the propanol had been removed. The resulting aqueous solution was spray dried to yield a pale yellow powder (280 g).

### EXAMPLE 5

### Preparation of Ti(TOPED)

292 g (1 mole) of N,N,N′,N′-tetrakis(2-hydroxypropyl)ethylenediamine (THPED) (from Alfa Aesar) was added to 284 g (1 mole) of tetra(isopropoxy)titanium (VERTEC™ TIPT, from Johnson Matthey Catalysts), with stirring, to give a clear solution. The isopropanol produced during the reaction was removed by rotary evaporation under reduced pressure to yield a white powder (336 g) of N,N,N′,N′-tetrakis(2-oxypropyl)ethylenediamine titanium (Ti(TOPED)). The white powder was analysed using ¹H-NMR and by elemental analysis.

The NMR analysis yielded the following chemical shift data (relative to tetramethyl silane (TMS), where m indicates a multiplet, which is consistent with the presence of N,N,N′,N′-tetrakis(2-oxypropyl)ethylenediamine titanium:

¹H NMR (400 MHz); 5.40-4.30 (4H, m), 3.60-2.30 (12H, m), 1.70-0.70 (12H, m).

The elemental analysis yielded the following data:

Found: C, 49.38; H, 8.53; N, 8.16%.

Theoretical for [Ti(TOPED)]₂: C, 50.01; H, 8.39; N, 8.33%.

Ti Content (wt %): Found: 14.12, Theoretical for Ti[TOPED]₂: 14.27.

### EXAMPLE 6

### Preparation of Ti[TOBED]

Tetraisopropyl titanate (28.422 g) was slowly added to N,N,N′,N′ tetra (2-hydroxybutyl)ethylenediamine (38.853 g) with constant mixing; heat was released. The resulting solution of N,N,N′,N′-tetrakis(butoxy)ethylenediamine titanium (Ti[TOBED]) was then diluted in diethylene glycol (16.73 g).

### EXAMPLE 7

The compound of Example 1 was dissolved in methanol to form a 10% w/w solution. The solution was boiled for one hour and then the methanol was removed by evaporation. The resulting pale yellow powder was found to be the same compound as the starting material, showing that the compound was stable to methanolysis under the conditions used.

### EXAMPLE 8

### Preparation of Zr(TOEED)

44.3 g of a solution of n-propyl zirconate in n-propyl alcohol (0.1 moles of zirconium) was slowly added to N,N,N′,N′ Tetra (hydroxy-2-ethyl)ethylenediamine (23.631 g) with constant mixing; heat was released. A colourless liquid resulted from which crystals precipitated on standing. The crystals were presumed to be dimeric, i.e. [Zr(TOEED)]₂.

### EXAMPLE 9

### Preparation of Zr(TOPED)

44.3 g of a solution of n-propyl zirconate in n-propyl alcohol (0.1 moles of zirconium) was slowly added to N,N,N′,N′ Tetra (hydroxy-2-propyl)ethylenediamine (29.242 g) with constant mixing; heat was released. A colourless liquid resulted from which crystals precipitated on standing.

### EXAMPLE 10

### Direct Esterification

Phthalic anhydride (148 g, 1.00 mole) and 2-ethyl-1-hexanol (315 g, 2.42 mole) were added to a 1-litre flask fitted with a suba seal, capillary tube and thermometer; magnetic stirrer bar. The reaction flask was fitted with a heating mantle and Dean and Stark apparatus to remove water as the reaction side product. A nitrogen inlet was then connected to the capillary tube. The catalyst, either TIPT (0.40 g, 1.41×10⁻³ mole) or Ti[TOEED] (0.40 g, 1.43×10⁻³ mole), was dissolved in 2-ethyl-1-hexanol (10 g, 0.08 mole) and added using a syringe to the reaction mixture at ambient temperature. The reaction flask was then heated on the highest setting of the mantle and the reaction timer was started. When the reaction mixture reached a temperature of 200±5° C., the vacuum was applied as necessary to maintain a fast distillation rate and the reaction temperature maintained at 200±5° C. Conversion was calculated from the acid value, determined by titration using 0.1N alcoholic KOH and bromo-thymol-blue indicator. The results are shown in Table 1.

### Comparative Method (Hot Addition)

The method above was repeated except that the TIPT catalyst in 2-ethyl-1-hexanol solution was added to the heated reaction mixture and then the reaction timer was started. This comparative method was intended to minimise the opportunity for the TIPT to become hydrolysed in contact with the water produced as a by-product of the reaction.

TABLE 1

| | % Conversion | | |
|---|---|---|---|
| Time (minutes) | Ti[TOEED] (cold) | TIPT (hot) | TIPT (cold) |
| 60 | 98.48 | — | — |
| 90 | 99.42 | 94.58 | 88.91 |
| 120 | 99.98 | 99.6 | 98.64 |
| 140 | — | 99.93 | 99.39 |
| 150 | — | 99.96 | 99.80 |

The results in Table 1 show Ti[TOEED] to be a more active Lewis acid catalyst than TIPT, in the direct-esterification of phthalic anhydride with 2-ethyl-1-hexanol to produce dioctylphthalate. The hydrolysis of titanium catalysts results in the formation of insoluble aggregates of titanium hydroxide type species, known to be low in catalytic activity. The higher hydrolytic stability of Ti[TOEED] compared with TIPT, accounts for the observed differences in catalytic activity in this reaction. The deactivation of TIPT by water, a side product of the direct-esterification reaction, is less when the catalyst is added after the reaction temperature has reached 180° C., (hot method) because of the removal of water produced during the initial stages of the reaction and of any water in the reactants. The effect of less hydrolysis is shown by the higher conversions using TIPT added to the hot mixture compared with the cold addition.

### EXAMPLE 11

### Transesterification

The transesterification of rapeseed oil with methanol to form biodiesel was carried out using a 1:6 molar ratio of tri-glyceride/methanol and catalysed by Ti[TOPED] (1.8% w/w based on tri-glyceride). A reaction mixture of rapeseed oil (220 g, 0.25 mole), methanol (48.0 g, 1.50 mole), and Ti[TOPED] (4.00 g, 1.19×10⁻² mole), was weighed into the glass liner of a Parr 4843 1-L autoclave, fitted with a overhead stirrer (300 rpm). The autoclave was sealed at room temperature before being purged with nitrogen three times. The reactor was heated for 90 minutes up to a temperature of 200° C. then allowed to cool. The resulting material was removed from the autoclave and placed in a separating funnel to allow the glycerol phase to separate, before the product was diluted with tetrahydrofuran (THF) for analysis by high performance liquid chromatography (HPLC). The HPLC analysis was performed on a Waters 2690 HPLC system, fitted with a UV-Vis detector, using HPLC-grade THF as the eluent. The total volume recovered was made up to 500 ml using HPLC-grade THF. A 10 ml aliquot of this was made up to 100 ml and used for the analysis. The HPLC was calibrated using standards for the tri-glyceride (rapeseed oil), di-glyceride, mono-glyceride and ester (biodiesel) and the results, shown in Table 2, are reported as percentages, which have been calculated from the peak size and normalised to give a 100% total. The reaction was repeated in the absence of any titanium catalyst, as a blank, for comparison.

TABLE 2

| Catalyst | Tri-glyceride | Di-glyceride | Mono-glyceride | Ester |
|---|---|---|---|---|
| none | 82.3 | 14.7 | 2.9 | 0 |
| Ti[TOPED] | 1.7 | 3 | 10.6 | 84.7 |

The Ti[TOPED] is shown to be an effective Lewis acid catalyst, for the trans-esterification reaction between methanol and tri-, di- and mono-glycerides, to produce the methyl ester (biodiesel) in high yield. The high activity of the catalyst is thought to be related its stability to methanolysis; with a catalyst of greater stability expected provide a greater catalytic activity. The methanolysis of titanium catalysts results in the formation of an array of insoluble aggregates of titanium methoxide type species which are known to be low in catalytic activity.

### EXAMPLE 12 & COMPARATIVE EXAMPLE 13

### Preparation of Polyethylene Terephthalate

Solid terephthalic acid (PTA) was charged to a reactor with monoethylene glycol (MEG) and catalyst. The temperature is ramped from 60° C. to 260° C. over a 90 minute time period, at 40 psi until all water has been removed (direct esterification). As the condensation reaction proceeds water is produced and evaporates together with some MEG. The MEG is separated in the distillation column and recycled back into the reactor, whilst the separated water is removed. The direct esterification time is measured as the time interval between the start of esterification (at approximately 210° C.) and the complete removal of water from the system. The resulting bis-hydroxy ethyl terephthalate (BHET) monomer formed in the first reaction stage, was then polymerised at 2 mbar pressure and 290° C. until the polymer had reached an intrinsic viscosity of 0.6 dl/g. As the condensation reaction proceeded, MEG and a small amount of water were produced and removed from the reactor. The polycondensation time is measured as the time between the start of the low pressure being applied and the target intrinsic viscosity being reached.

The results, shown in Table 3, demonstrate that Ti[TOEED] is an active Lewis acid catalyst in the direct-esterification of terephthalic acid with ethylene glycol to produce bis-hydroxy ethyl terephthalate and the polycondensation of bis-hydroxy ethyl terephthalate to produce polyethylene terephthalate. The high hydrolytic stability of Ti[TOEED], allows it to maintain its catalytic activity in the polycondensation reaction and produce a relatively fast reaction.

TABLE 3

| | | | | Direct | |
|---|---|---|---|---|---|
| | | Ti or Sb | MEG:PTA | Esterification | Polycondensation |
| Example | Catalyst | (ppm) | (mol:mol) | Time (min) | Time (min) |
| 12 | Ti[TOEED] | 8 | 1.2 | 94 | 88 |
| 13 | Antimony | 250 | 1.2 | 85 | 97 |
| (Comparative) | acetate | | | | |

### EXAMPLE 14

### N-oxidation of aniline using 1 Ti[TOEED]:100 Aniline:160H₂O₂

2Ph-NH₂+3H₂O₂→1 Ph-N═N⁺(O⁻)-Ph+5H₂O

The reaction was carried out using a small (approx 6%) excess over the stoichiometric amount of hydrogen peroxide in aqueous solution as described below. The excess H₂O₂ was provided in order to compensate for any decomposition of hydrogen peroxide which may take place during the set up of the reaction.

Ti[TOEED] (15.1 mg, 53.9 μmol) was dissolved in demineralised water (25.0 ml) and added to a glass vial containing aniline (500 mg, 5.38 mmol) and a magnetic stirrer bar. Hydrogen peroxide, approx 35% in water (840 mg, 8.65 mmol), was dissolved in demineralised water (25.0 ml) and added to the glass vial. The reaction mixture was stirred at ambient temperature, with cooling from a water bath, for 2 hrs. The reaction mixture immediately turned into a bright yellow homogeneous solution upon addition of the hydrogen peroxide solution. The solution developed a darker red-brown colouration, with dark coloured inhomogeneous droplets during the progression of the reaction.

The aqueous reaction mixture was extracted with ethyl acetate (3×50 ml), to leave a clear pale yellow solution. The dark red/brown organics were dried over magnesium sulphate and filtered. The organic solvent was removed on a rotary evaporator to yield a dark red/brown semi-solid. The samples were subjected to gas chromatography mass spectrometry (GC-MS) electron impact (EI⁺) analysis for the identification of the reaction products and gas chromatography (GC) flame ionisation detection (FID) for quantitative analysis of the reaction products. The compounds found in the reaction product mixture were: nitrosobenzene, aniline, nitrobenzene, azobenzene, azoxybenzene and an unidentified product eluted after the others. The peak areas, normalised to 100%, are shown in Table 4, together with the aniline conversion and selectivity of aniline conversion to azoxybenzene. The results using Ti[TOEED] as catalyst show a high conversion level of aniline into azoxybenzene, using a stoichiometric equivalence of hydrogen peroxide, low levels of catalyst (100 aniline:1 Ti), in only 2 hours. The selectivity of the reaction towards azoxybenzene formation over azobenzene formation (84:1, respectively) is relatively high considering the short reaction time. The selectivity of the reaction towards azoxybenzene formation, based on aniline conversion, is about 97%.

### EXAMPLE 15

### Comparison

Example 14 was repeated but using as a catalyst triethanolaminetitanate (VERTEC™ TET) as a comparison. The very low conversion level of aniline into azoxybenzene (<4%) using TET indicates that the catalyst has undergone deactivating hydrolysis reactions. This has also resulted in poor reaction selectivity. The selectivity of the reaction towards azoxybenzene formation over azobenzene formation is 4:1, respectively. The selectivity of the reaction towards azoxybenzene formation, based on aniline conversion, is about 57%.

### EXAMPLE 16

### Higher Reactant Concentration 1 Ti[TOEED]:100 Aniline:160H₂O₂

Ti[TOEED] (151 mg, 539 mmol) was dissolved in demineralised water (25.0 ml) and added to a glass vial containing aniline (5.00 g, 53.9 mmol) and a magnetic stirrer bar. Hydrogen peroxide, ˜35% in water (8.40 g, 86.5 mmol), was dissolved in demineralised water (25.0 ml) and added to the glass vial. The reaction mixture was stirred at ambient temperature, with cooling from a water bath, for 2 hrs. The reaction mixture immediately turned into a bright yellow homogeneous solution upon addition of the hydrogen peroxide solution. The solution developed a darker red-brown colouration, with dark coloured inhomogeneous droplets during the progression of the reaction.

The aqueous reaction mixture was extracted and analysed as described in Example 12. The results show a high conversion level of aniline into azoxybenzene (about 90%), using a stoichiometric equivalence of hydrogen peroxide, low levels of catalyst (100 aniline:1 Ti), in only 2 hours. This reaction was undertaken at a relatively high concentration (5.0 g aniline in 50 ml water) compared with Example 14. The selectivity of the reaction towards azoxybenzene formation over azobenzene formation is 225:1. The selectivity of the reaction towards azoxybenzene formation, based on aniline conversion, is 94%.

### EXAMPLE 17

### Comparison 1 TET:100 Aniline:160H₂O₂

The reaction was carried out as described in Example 16, using the same high concentration of reactants in solution but using VERTEC TET (314 mg, 539 μmol) as a catalyst instead of TI[TOEED]. The conversion level of aniline into azoxybenzene (about 39%) using TET indicates that the catalyst has undergone partial deactivation via hydrolysis reactions. The selectivity of the reaction towards azoxybenzene formation over azobenzene formation is 35:1, respectively. The selectivity of the reaction towards azoxybenzene formation, based on aniline conversion, is 95%.

### EXAMPLE 18

### 1 Ti[TOEED]:500 Aniline:800H₂O₂

Ti[TOEED] (3.02 mg, 10.8 μmol) was dissolved in demineralised water (25.0 ml) and added to a glass vial containing aniline (500 mg, 5.39 mmol) and a magnetic stirrer bar. A 35% solution of hydrogen peroxide in water (840 mg, 8.65 mmol), was dissolved in demineralised water (25.0 ml) and added to the glass vial. The reaction mixture was stirred at ambient temperature, with cooling from a water bath, for 24 hrs. The reaction mixture immediately turned into a bright yellow homogeneous solution upon addition of the hydrogen peroxide solution. The solution developed a darker red-brown colouration, with dark coloured inhomogeneous droplets during the progression of the reaction.

The aqueous reaction mixture was extracted and analysed as described in Example 14. The results show a 96.6% conversion of aniline into azoxybenzene using a stoichiometric equivalence of hydrogen peroxide and very low levels of catalyst (500 aniline:1 Ti). The selectivity towards azoxybenzene formation over azobenzene formation is >1000:1.

### EXAMPLES 19-21

### 1 Ti[TOEED]:100 Aniline:160H₂O₂

Ti[TOEED] (75.5 mg, 269 mmol) was dissolved in demineralised water (25.0 ml) and added to a glass vial containing aniline (2.50 g, 26.9 mmol) and a magnetic stirrer bar. Hydrogen peroxide, ˜35% in water (4.20 g, 43.2 mmol), was dissolved in demineralised water (25.0 ml) and added to the glass vial. The reaction mixture was stirred at ambient temperature, with cooling from a water bath, for 3 hrs. The reaction mixture immediately turned into a bright yellow homogeneous solution upon addition of the hydrogen peroxide solution. The solution developed a darker red-brown colouration, with dark coloured inhomogeneous droplets during the progression of the reaction.

The aqueous reaction mixture was extracted and analysed as described in Example 14.

The clear pale yellow aqueous layer was reused in two subsequent reactions, with further additions of aniline (2.50 g, 26.9 mmol) and hydrogen peroxide 35% solution (4.20 g, 43.2 mmol). Following each reaction, the reaction product mixture was extracted and the organic layer was analysed by the GC-MS and GC methods of Example 14. The two subsequent reactions are shown as Examples 20 and 21 in Table 4. The loss of activity (conversion of aniline) between subsequent batch reactions is believed to be due to a gradual loss of catalyst from the aqueous layer, with each ethyl acetate wash. The selectivity of the reactions towards azoxybenzene formation, based on aniline conversion, increases with each subsequent batch; Example 19=95%, Example 20=96%, Example 21=98%.

### EXAMPLE 22

### Synthesis of N,N,N′,N′-tetrakis(2-oxypropyl)ethylenediamine tin

3.55 g (1.22×10⁻² mole) of N,N,N′,N′-tetrakis(2-hydroxypropyl)ethylenediamine (THPED) was added to 5.0 g (1.22×10⁻² mole) of tin tetrabutoxide, in dichloromethane (25 mL), with stirring, to give a clear solution. An exotherm was observed during the addition of the THPED. The dichloromethane and butanol produced during the reaction was removed by rotary evaporation under reduced pressure to yield a white solid. The solid was washed with hexanes, filtered and dried to give N,N,N′,N′-tetrakis(2-oxypropyl)ethylenediamine tin, as a white powder (4.9 g, 1.21×10⁻² mole).

### EXAMPLE 23

### Synthesis of N,N,N′,N′-tetrakis(2-oxyethyl)ethylenediamine tin

2.87 g (1.22×10⁻² mole) of N,N,N′,N′-tetrakis(2-hydroxyethyl)ethylenediamine (THEED) was added to 5.0 g (1.22×10⁻² mole) of tin tetrabutoxide, in dichloromethane (25 mL), with stirring, to give a clear solution. An exotherm was observed during the addition of the THEED. The dichloromethane and butanol produced during the reaction was removed by rotary evaporation under reduced pressure to yield a white solid. The solid was washed with hexanes, filtered and dried to give N,N,N′,N′-tetrakis(2-oxyethyl)ethylenediamine tin, as a white powder (4.2 g, 1.21×10⁻² mole).

### EXAMPLE 24

### Synthesis of Al[(OCH₂CH₂)₂NCH₂CH₂N(CH₂CH₂O)(CH₂CH₂OH)]

0.287 g (1.22×10⁻³ mole) of N,N,N′,N′-tetrakis(2-hydroxyethyl)ethylenediamine (THEED) was added to 0.30 g (1.22×10⁻³ mole) of aluminium tri-sec-butoxide, with stirring, to give a clear solution. An exotherm was observed during the addition of the THEED. The dichloromethane and sec-butanol produced during the reaction was removed by rotary evaporation under reduced pressure to yield a yellow liquid (0.3 g, 1.20×10⁻³ mole).

TABLE 4

| | | | | | | | | | Conversion | |
|---|---|---|---|---|---|---|---|---|---|---|
| Ex- | | Catalyst:aniline:H₂O₂ | GC peak area % | | | | | | of aniline | Selectivity |
| ample | Catalyst | (mol:mol) | Nitrosobz | Aniline | Nitrobz | Azobz | Azoxybz | Unknown | (%) | (%) |
| 14 | Ti[TOEED] | 1:100:160 | 0.4 | 4.6 | 0.7 | 1.1 | 92.7 | 0.5 | 95.4 | 97 |
| 15* | TET | 1:100:160 | 1.1 | 93.1 | 1.0 | 0.9 | 3.9 | 0 | 6.9 | 57 |
| 16 | Ti[TOEED] | 1:100:160 | 2.8 | 4.4 | 1.7 | 0.4 | 89.8 | 0.9 | 95.6 | 94 |
| 17* | TET | 1:100:160 | 0.1 | 59.3 | 0.8 | 1.1 | 38.6 | 0.1 | 40.7 | 95 |
| 18 | Ti[TOEED] | 1:500:800 | 0.5 | 0.0 | 2.1 | 0.1 | 96.6 | 0.7 | 100 | 97 |
| 19 | Ti[TOEED] | 1:100:160 | 1.2 | 2.5 | 1.2 | 0.6 | 93.2 | 1.3 | 97.5 | 95 |
| 20 | Ti[TOEED] | 1:100:160 | 1.0 | 4.2 | 0.7 | 0.4 | 91.8 | 0.7 | 95.8 | 96 |
| 21 | Ti[TOEED] | 1:100:160 | 0.1 | 20.8 | 0.4 | 0.5 | 78.0 | 0.2 | 79.2 | 98 |

*comparative example.
Nitrosobz = nitrosobenzene,
Nitrobz = nitrobenzene,
Azobz = azobenzene,
Azoxybz = azoxybenzene

1. A method of carrying out a catalysed reaction comprising carrying out the reaction in the presence of a catalyst comprising a metal-organic compound having the following formula:
M(HO(CR¹R²)_{z})ₐ(O(CR¹R²)_{z})_{b}Y—(CR³R⁴)ₓ—Y((CR¹R²)_{z}O)_{c}((CR¹R²)_{z}OH)_{d}.nR⁵OH (Formula I)
in which:
M is a metal atom;
Y is selected from P and N;
each R¹, R², R³ and R⁴ is independently selected from H, alkyl, aryl, substituted alkyl or substituted aryl;
R⁵ is hydrogen, an alkyl group, a hydroxy-functionalised alkyl group, a polyoxyalkyl moiety, R⁶O or R⁷COO where R⁶ and R⁷ may each represent H, alkyl, aryl or alkyl-aryl;
d and a are each 0 or 1;
b and c are each 1 or 2;
b+c=the valency of M;
a+b+c+d=4;
each z is independently 1, 2, 3 or 4;
x represents the least number of C atoms between the Y atoms and is 2 or 3; and
n is a number in the range from 0 to 4;
wherein said catalysed reaction is selected from the group consisting of an aldol reaction, Michael addition, Mannich reaction, ether formation, oxidation, oxidative coupling, peptide synthesis, amide synthesis and Claisen reaction.

2. The method according to claim 1, wherein Y represents a nitrogen atom.

3. The method according to claim 1, wherein M comprises titanium, zirconium, hafnium, or iron(III).

4. The method according to claim 1, wherein each of R³ and R⁴ is H.

5. The method according to claim 1, wherein each of R¹ and R² is selected from the group consisting of H, a methyl group and an ethyl group.

6. The method according to claim 1, wherein each z is 2 or 3.

7. The method according to claim 6 wherein the metal-organic compound comprises the reaction product of a metal compound with one of N,N,N′,N′-tetrakis(2-hydroxyethyl)ethylenediamine, N,N,N′,N′-tetrakis(2-hydroxypropyl)ethylenediamine or N,N,N′,N′-tetrakis(2-hydroxybutyl)ethylenediamine.

8. The method according to claim 1 wherein water or an alcohol is present in the reaction mixture.

9. The method according to claim 8, wherein the reaction mixture contains from 0.01-70% by weight of said metal-organic compound and from 99-0.1% of water in addition to organic compounds.

10. The method according to claim 1, wherein n=1 or 2.

11. The method according to claim 1, wherein the catalysed reaction comprises a reaction to form one or more single or multiple bonds between carbon and carbon, carbon and oxygen, carbon and nitrogen, nitrogen and oxygen, sulphur and oxygen and/or nitrogen and nitrogen atoms.

12. (canceled)

13. The method according to claim 12 comprising the oxidation or oxidative coupling of a chemical substrate by contacting the chemical substrate with
(i) an oxidising agent selected from the group consisting of hydrogen peroxide, an organic hydroperoxide and -peroxyacids; and
(ii) said metal organic compound;
under conditions of temperature and pressure suitable to effect the desired reaction.

14. The method according to claim 13, wherein said chemical substrate comprises a compound of the type selected from the group consisting of an alkene, an alkyne, a carboxylic acid, a sulphur-containing compound, H₂S, organic sulphide, a cyclic sulphur compound, wood pulp, textile, a hypochlorite, a nitrile, a nitrite, an amine, hydroxylamine, nitroso compound, azoxy compound and a nitrone.

15. A composition comprising:
(a) from 0.01-70% by weight of a metal-organic compound of Formula I:
M(HO(CR¹R²)_{z})ₐ(O(CR¹R²)_{z})_{b}Y—(CR³R⁴)ₓ—Y((CR¹R²)_{z}O)_{c}((CR¹R²)_{z}OH)_{d}.nR⁵OH (Formula I)
in which:
M is a metal atom;
Y is selected from P and N;
each R¹, R², R³ and R⁴ is independently selected from H, alkyl, aryl, substituted alkyl or substituted aryl;
R⁵ is hydrogen, an alkyl group, a hydroxy-functionalised alkyl group, a polyoxyalkyl moiety, R⁶O or R⁷COO where R⁶ and R⁷ may each represent H, alkyl, aryl or alkyl-aryl;
d and a are each 0 or 1;
b and c are each 1 or 2;
b+c=the valency of M;
a+b+c+d=4;
each z is independently 1, 2, 3 or 4;
x represents the least number of C atoms between the Y atoms and is 2 or 3; and
n is a number in the range from 0 to 4;
(b) from 1-99.99% of water or an alcohol; and
(c) from 1-50% of an oxidising agent selected from the group consisting of hydrogen peroxide, an organic hydroperoxide and -peroxyacids.

16. (canceled)

17. The composition according to claim 15, wherein Y represents a nitrogen atom.

18. The composition according to claim 15, wherein M comprises titanium, zirconium, hafnium, or iron(III).

19. The composition according to claim 15, wherein each of R³ and R⁴ is H.

20. The composition according to claim 15, wherein each of R¹ and R² is selected from the group consisting of H, a methyl group and an ethyl group.

21. The composition according to claim 15, wherein each z is 2 or 3.

22. The composition according to claim 21 wherein the metal-organic compound comprises the reaction product of a metal compound with one of N,N,N′,N′-tetrakis(2-hydroxyethyl)ethylenediamine, N,N,N′,N′-tetrakis(2-hydroxypropyl)ethylenediamine or N,N,N′,N′-tetrakis(2-hydroxybutyl)ethylenediamine.

23. The method according to claim 13, wherein said catalysed reaction comprises the oxidative coupling of an aromatic amine to form an azoxy compound.

24. The method according to claim 13, said catalysed reaction comprises the oxidative coupling of an aromatic amine to form a colored compound which is useful for dyeing or coloring fibres, cloth, human skin, human hair, animal skin or animal hair.
